SEQUENCE LISTING (16 sequences)

SEQ ID NO: 1 - protein, 458 aa, Oryctolagus cuniculus
METGLRWLLLVAVLKGVQCQSVEESGGRLVTPGTPLTLTCTVSGIDLSDYAMGWVRQAPGKGLEYIGIISSSGNTHYARWARGRFTISKTSSTTVDLKMTSLTTEDTATYFCARNYPGYANYALWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 2 - protein, 237 aa, Oryctolagus cuniculus
MDTRAPTQLLGLLLLWLPGATFARIVMTQTPASVSAAVGGTVTIKCQASESISNWLSWYQQKPGQPPKLLIYRASTLASGVPSRFSGSGSGTEYTLTISDLECADAATYYCQQDYIYNDIDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 3 - protein, 468 aa, Oryctolagus cuniculus
METGLRWLLLVAVLKGVQCQSLEESGGDLVKPGASLTLTCTASGFSFSSSAYMCWVRQAPGKGLEWIACIAAGSVGRTAYASWAKGRFTLSKISSTTVTLQMTSLTAADTATYFCARGYAAYGGYDWPITYFKLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 4 - protein, 238 aa, Oryctolagus cuniculus
MDTRAPTQLLGLLLLWLPGARCADIVMTQTPASVEAAVGGTVTIKCQASQSIYSGLAWYQQKPGQPPKLLIYDASDLPSGVPSRFKGSGSGTEFTLTISDLECADAATYYCQSYYGSSTTYGNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 5 - protein, 5 aa, Oryctolagus cuniculus
DYAMG

SEQ ID NO: 6 - protein, 16 aa, Oryctolagus cuniculus
IISSSGNTHYARWARG

SEQ ID NO: 7 - protein, 10 aa, Oryctolagus cuniculus
NYPGYANYAL

SEQ ID NO: 8 - protein, 11 aa, Oryctolagus cuniculus
QASESISNWLS

SEQ ID NO: 9 - protein, 7 aa, Oryctolagus cuniculus
RASTLAS

SEQ ID NO: 10 - protein, 12 aa, Oryctolagus cuniculus
QQDYIYNDIDNA

SEQ ID NO: 11 - protein, 6 aa, Oryctolagus cuniculus
SSAYMC

SEQ ID NO: 12 - protein, 18 aa, Oryctolagus cuniculus
CIAAGSVGRTAYASWAKG

SEQ ID NO: 13 - protein, 17 aa, Oryctolagus cuniculus
GYAAYGGYDWPITYFKL

SEQ ID NO: 14 - protein, 11 aa, Oryctolagus cuniculus
QASQSIYSGLA

SEQ ID NO: 15 - protein, 7 aa, Oryctolagus cuniculus
DASDLPS

SEQ ID NO: 16 - protein, 13 aa, Oryctolagus cuniculus
QSYYGSSTTYGNT

(19) United States
(12) Patent Application Publication
(10) Pub. No.: US 2024/0210403 A1
(43) Pub. Date: Jun. 27, 2024

(54) LATERAL FLOW ANALYSIS AND BREAST CANCER

(71) Applicant: Martell Diagnostic Laboratories Inc., Roseville, MN (US)

(72) Inventors: Franklin Pass, Edina, MN (US); Steven Stoesz, Minneapolis, MN (US); Justin Lengfeld, Brooklyn Park, MN (US)

(21) Appl. No.: 18/555,643

(22) PCT Filed: Apr. 15, 2022
(86) PCT No.: PCT/US2022/071747

§ 371 Date: Oct. 16, 2023

Related U.S. Application Data
(60) Provisional application No. 63/201,193, filed on Apr. 16, 2021.

(51) Int. Cl. G01N 33/574; G01N 33/543

(52) U.S. Cl. CPC G01N 33/57415; G01N 33/54388; G01N 2333/71

(57) ABSTRACT

Provided herein is a point-of-care multiple diagnostic assay with multiple test lines allowing the rapid and simultaneous detection of multiple analytes present in samples, including, for example, HER2 positive hyperproliferative disorders such as a neoplastic disorders.

### BACKGROUND

Over the last two decades, breast cancer patients in developed countries have experienced significant increases in survival, especially women with tumors that overexpress the growth factor protein HER2. These HER2-positive women are treated with drugs that disable the HER2 protein (HER2 targeted drugs). Currently, candidates for HER2 targeted drugs are selected using biomarker tissue tests performed on biopsy tissue samples. Tests can be used to monitor HER2 targeted therapy once patients are selected for treatment. The blood test is performed on blood serum samples.

The HER2 targeted drugs, patient selection, and even current blood testing is expensive. These benefits have not extended to markets such as Honduras, Bangladesh, or sub-Saharan Africa, where only 10% of women can afford the biomarker tissue testing or targeted drugs. Additionally, the sub-Saharan Africa healthcare systems, and many others, lack the pathology resources to manage tissue biomarker testing, and therefore, most breast cancer patients receive the least expensive but often not the best treatment.

### SUMMARY

Breast cancer treatment has improved considerably over the past 20 years as a result of, for example, earlier detection, better surgical techniques, a variety of new drugs, and new imaging methods (e.g., to detect recurrence). Provided herein is more a affordable diagnostic method using lateral flow assay, so as to meet the needs of the healthcare systems across the world.

HER2/neu is a member of the human epidermal growth factor receptor (HER/EGFR/ERBB) family. Amplification or over-expression of this oncogene has been shown to play a role in the development and progression of certain aggressive types of breast cancer. The protein has become a biomarker and target of therapy for approximately 30% of breast cancer patients.

The HER2/neu protein is proteolytically cleaved by membrane-associated serine proteases to release the extracellular domain, which then can be detected and measured in bodily fluids. A HER2/neu in vitro diagnostic (IVD) immunoassay for blood was introduced (as described in, for example, Carney et al., 2003, Clin. Chem., 49(10): 1579-98), but the results were often confusing, and the test fell into clinical disuse.

One embodiment provides a method to detect HER2 polypeptides or fragments thereof in a sample comprising: (a) analyzing a biological sample using a lateral flow immunoassay (LFA), wherein the LFA comprises at least one anti-HER2 rabbit monoclonal antibody or binding fragment thereof selected from the group consisting of: (i) an anti-HER2 rabbit monoclonal antibody comprising a heavy chain variable domain (VH) comprising the amino acid sequence of SEQ ID NO: 1 and a light chain variable domain (VL) comprising the amino acid sequence of SEQ ID NO:2; (ii) an anti-HER2 rabbit monoclonal antibody comprising a heavy chain variable domain (VH) comprising the amino acid sequence of SEQ ID NO:3 and a light chain variable domain (VL) comprising the amino acid sequence of SEQ ID NO:4; (iii) an anti-HER2 rabbit monoclonal antibody or binding fragment thereof comprising a heavy chain and a light chain, wherein the heavy chain comprises three CDR regions having the amino acid sequence SEQ ID NO: 5, 6 and 7; and the light chain comprises three CDR regions having the amino acid sequence SEQ ID NO: 8, 9 and 10 and (iv) an anti-HER2 rabbit monoclonal antibody or binding fragment thereof comprising a heavy chain and a light chain, wherein the heavy chain comprises three CDR regions having the amino acid sequence SEQ ID NO: 11, 12 and 13; and the light chain comprises three CDR regions having the amino acid sequence SEQ ID NO: 14, 15 and 16 and (b) detecting polypeptide/antibody complexes, wherein the detection of polypeptide/antibody complexes is an indication that the HER2 polypeptide is present in the sample.

In one embodiment, the sample is lymph node or tissue aspirate (e.g., breast), serum, whole blood, plasma, urine, saliva, tears, cerebrospinal fluid, supernatant from normal cell lysates, supernatant from pre-neoplastic cell lysates, supernatant from neoplastic cell lysates and/or supernatants from carcinoma cell lines maintained in tissue culture.

In another embodiment, the lateral flow assay comprises a detectable label, wherein the label is detectable by visual inspection. In one embodiment, the label detectable by visual inspection comprises gold colloidal particles.

In one embodiment, the sample is obtained from a subject that is being treated with a therapeutic agent, such as at least one of trastuzumab, trastuzumab emtansine, pembrolizumab, pertuzumab, nivolumab, atezolizumab or a combination thereof. Another embodiment further comprises (c) determining how a patient is responding to treatment based on the results in (b).

In another embodiment more than one, such as multiple, analytes are detected. In addition to HER2, other analytes that can be detected on the same lateral flow assay include a small molecule drug or therapeutic agent, a cancer antigen, an antibody (e.g., an antibody in response to a bacterial or viral infection or a treatment antibody), nucleic acids and/or proteins.

Provided herein are recombinant rabbit monoclonal antibodies which bind with specificity to HER2, such as to the extracellular domain of HER2.

One embodiment provides an anti-HER2 rabbit monoclonal antibody comprising a heavy chain variable domain (VH) comprising the amino acid sequence of SEQ ID NO: 1 and a light chain variable domain (VL) comprising the amino acid sequence of SEQ ID NO:2.

Another embodiment provides an anti-HER2 rabbit monoclonal antibody comprising a heavy chain variable domain (VH) comprising the amino acid sequence of SEQ ID NO:3 and a light chain variable domain (VL) comprising the amino acid sequence of SEQ ID NO:4.

Another embodiment provides an anti-HER2 rabbit monoclonal antibody or binding fragment thereof comprising a heavy chain and a light chain, wherein: (i) the heavy chain comprises three CDR regions having the amino acid sequence SEQ ID NO: 5, 6 and 7; and (ii) the light chain comprises three CDR regions having the amino acid sequence SEQ ID NO: 8, 9 and 10.

One embodiment provides an anti-HER2 rabbit monoclonal antibody or binding fragment thereof comprising a heavy chain and a light chain, wherein: (i) the heavy chain comprises three CDR regions having the amino acid sequence SEQ ID NO: 11, 12 and 13; and (ii) the light chain comprises three CDR regions having the amino acid sequence SEQ ID NO: 14, 15 and 16.

In one embodiment, the antibody is conjugated to a detection agent.

One embodiment provides a composition comprising one or more antibodies described herein and a carrier,

One embodiment provides a method to detect HER2 polypeptides or fragments thereof in a sample comprising: (a) contacting the anti-HER2 rabbit monoclonal antibody or binding fragment thereof of any one of claims 1-6 with a test sample under conditions that allow polypeptide/antibody complexes to form; and (b) detecting polypeptide/antibody complexes, wherein the detection of polypeptide/antibody complexes is an indication that the HER2 polypeptide is present in the sample.

Another embodiment provides a method to monitor HER2 polypeptides or fragments thereof in a sample from a subject comprising: (a) contacting at least one of the anti-HER2 rabbit monoclonal antibodies or binding fragments thereof of any one of claims 1-6 with said sample under conditions that allow polypeptide/antibody complexes to form; (b) detecting polypeptide/antibody complexes, wherein the detection of polypeptide/antibody complexes indicates HER2 polypeptides or fragments thereof are present in the subject; and (c) performing steps (a) and (b) at a plurality of time points so as monitor HER2 polypeptides or fragments thereof in said subject over time. In one embodiment, the sample is contacted in (a) with: (i) a capture antibody or a binding fragment thereof, and (ii) a detection antibody or a functional fragment thereof. In one embodiment the capture and detection antibodies bind to HER2. In another embodiment, the detection antibody binds to the capture antibody and/or the capture antibody is immobilized. In embodiment, the detection antibody comprises a detection agent. In one embodiment, the subject is being treated with trastuzumab, trastuzumab emtansine, pembrolizumab, nivolumab, atezolizumab or a combination thereof, wherein the trastuzumab, trastuzumab emtansine, pembrolizumab, nivolumab, atezolizumab or a combination thereof does not interfere or only moderately interferes with the binding of the capture and/or detection antibodies or binding fragments thereof.

In one embodiment, the sample is lymph node or tissue aspirate (e.g., breast), serum, whole blood, plasma, urine, saliva, tears, cerebrospinal fluid, supernatant from normal cell lysates, supernatant from pre-neoplastic cell lysates, supernatant from neoplastic cell lysates and/or supernatants from carcinoma cell lines maintained in tissue culture.

### BRIEF DESCRIPTION OF THE FIGURES

FIG. 1 shows a representative standard curve for the serum HER2 ELISA based on the data shown in Table 1.

FIG. 2 shows the reference range for the serum HER2 ELISA (see also Table 13).

FIG. 3 is a graph showing drug interference with 1B7/1G5-biotin antibody pair.

FIG. 4 graphically represents the data provided in Table 18.

FIG. 5 graphically represents the data provided in Table 19.

### DETAILED DESCRIPTION OF THE INVENTION

The practice of the methods and compositions described herein may employ, unless otherwise indicated, conventional techniques of pharmaceutical chemistry, molecular biology, drug formulation techniques, dosage regimes, immunology and biochemistry, all of which are within the skill of those who practice in the art.

Provided herein are recombinant rabbit monoclonal antibodies which bind with specificity to HER2, such as to the extracellular domain of HER2. The antibodies described herein provide several improvements over other anti-HER2 antibodies, in addition to their binding specificity, they exhibit little to no interference with therapeutic agents, making an immunoassay based on the antibodies provided herein more valuable in terms of providing much needed accurate information to the doctor/patient.

Substances that alter the measurable concentration of the analyte or alter antibody binding can potentially result in immunoassay interference. Interfering substances may lead to falsely elevated or falsely low analyte concentration in one or more assay systems depending on the site of the interference in the reaction. Interference in immunoassay may lead to the misinterpretation of a patient's results by the laboratory and the wrong course of treatment being given by the physician. For example, pertuzumab is one of the most common therapies used in the treatment of breast cancer to lower the level of HER2 in serum; unfortunately, pertuzumab interferes with many of the diagnostic assays that are currently used in the clinic, which drastically reduces the reliability of those assays. Interference of a diagnostic assay by a therapeutic antibody can take place at any number of stages of the assay or with any of the components involved in the assay (e.g., the capture antibody, the detection antibody). Significantly, pertuzumab, trastuzumab, margetuximab, and/or HER2 small molecule inhibitors (e.g., lapatinib, neratinib) exhibit little to no interference with the antibodies/immunoassay described herein, which allows for greater accuracy when testing and/or monitoring patients that are being administered a therapeutic regimen for HER2 positive breast cancer.

### Definitions

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular embodiments only and is not intended to be limiting of the invention. Unless defined otherwise, all technical and scientific terms used herein have the same meaning as commonly understood by one of ordinary skill in the art to which this invention belongs. The following definitions are intended to aid the reader in understanding the present invention but are not intended to vary or otherwise limit the meaning of such terms unless specifically indicated.

As used herein, the indefinite articles “a”, “an” and “the” should be understood to include plural reference unless the context clearly indicates otherwise. Thus, for example, reference to “an inhibitor” refers to one or more agents with the ability to inhibit a target molecule, and reference to “the method” includes reference to equivalent steps and methods known to those skilled in the art, and so forth.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the term “about” means plus or minus 10% of the indicated value. For example, about 100 means from 90 to 110.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

Transmembrane protein HER2 (human epidermal growth factor receptor 2) or HER2/neu is also known as receptor tyrosine-protein kinase erbB-2, CD340 (cluster of differentiation 340), proto-oncogene Neu, Erbb2 (rodent), or ERBB2 (human); a protein that in humans is encoded by the ERBB2 (erythroblastic oncogene B) gene. The HER2 protein has a molecular weight of about 185 kiloDaltons (kDa) and consists of an intracellular tyrosine kinase domain, a transmembrane domain and an extracellular domain.

Amplification, also known as the over-expression of the ERBB2 gene, occurs in approximately 15-30% of breast cancers, also known as HER2 positive breast cancer. HER2-positive breast cancer is a breast cancer that tests positive for a protein called human epidermal growth factor receptor 2 (HER2). This protein promotes the growth of cancer cells.

In about 1 of every 5 breast cancers, the cancer cells have extra copies of the gene that makes the HER2 protein. HER2-positive breast cancers tend to be more aggressive than other types of breast cancer. It is associated with increased disease recurrence and a poor prognosis; however, drug agents targeting HER2 in breast cancer have significantly positively altered the otherwise poor-prognosis natural history of HER2-positive breast cancer. It is recommended that every invasive breast cancer be tested for the presence of HER2 because the results significantly impact treatment recommendations and decisions.

As used herein, “detecting” refers to the action or process of identifying the presence of that which is being detected, such as HER2/neu in a sample. As used herein, the term “sample” is defined as blood, serum, plasma, urine, saliva, tears, cerebrospinal fluid, supernatant from normal cell lysates, supernatant from pre-neoplastic cell lysates, supernatant from neoplastic cell lysates, supernatants from carcinoma cell lines maintained in tissue culture, and breast aspirates or biopsies. Thus, any number of biological samples can be used in the immunoassays described herein including, without limitation, blood, serum, plasma, urine, saliva, tears, cerebrospinal fluid, supernatant from cell lysates (e.g., normal cells, pre-neoplastic cells, neoplastic cells, carcinoma cells), or breast aspirates or biopsies.

As used herein, “monitoring” refers to the action or process of identifying the presence of that which is being detected at least twice over a period of time.

The term “antibodies” refers to an intact antibody or an antigen-binding portion or fragment thereof that competes with the intact antibody for antigen binding. The term “antibodies” also includes any type of antibody molecule or specific binding molecule that specifically binds HER2. The terms “antigen-binding portion” of an antibody, “antigen-binding fragment” of an antibody, and the like, as used herein, include any naturally occurring, enzymatically obtainable, synthetic, or genetically engineered polypeptide, glycoprotein, or immunoglobulin that specifically binds to HER2 protein. Antigen-binding fragments of an antibody may be derived, e.g., from full antibody molecules using any suitable standard techniques such as proteolytic digestion or recombinant genetic engineering techniques involving the manipulation and expression of nucleic acids encoding antibody variable and optionally constant domains.

A monoclonal antibody is an antibody obtained from a group of substantially homogeneous antibodies. A group of substantially homogeneous antibodies can contain a small amount of mutants or variants. Monoclonal antibodies are highly specific and interact with a single antigenic site. Each monoclonal antibody typically targets a single epitope, while polyclonal antibody populations typically contain various antibodies that target a group of diverse epitopes. Monoclonal antibodies can be produced by many methods including, for example, hybridoma methods (Kohler and Milstein, Nature 256:495, 1975), recombination methods (U.S. Pat. No. 4,816,567), and isolation from phage antibody libraries (Clackson et al., Nature 352:624-628, 1991; Marks et al., J. Mol. Biol. 222:581-597, 1991).

The terms “subject,” “mammal” and “mammalian subject” as used herein refers to any animal classified as a mammal, including humans, higher non-human primates, rodents, and domestic and farm animals, such as cows, horses, dogs, and cats. In some embodiments of the invention, the mammal is a human (male or female).

As used herein, the terms “including”, “includes”, “having”, “has”, “with”, or variants thereof, are intended to be inclusive similar to the term “comprising.”

As used herein, said “contain”, “have” or “including” include “comprising”, “mainly consist of”, “basically consist of” and “formed of”; “primarily consist of”, “generally consist of” and “comprising of” belong to generic concept of “have” “include” or “contain”.

The terms “comprises,” “comprising,” and the like can have the meaning ascribed to them in U.S. Patent Law and can mean “includes,” “including” and the like. As used herein, “including” or “includes” or the like means including, without limitation.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### Rabbit Antibodies/Polypeptides

Rabbit monoclonal antibodies are a useful for many applications, including immunofluorescence, immunohistochemistry, flow cytometry, western blot, and ELISA assays. Compared to other animal models (e.g., mouse and rat), rabbits provide a better system for monoclonal antibody production because the rabbit immune system responds to a broader range of antigens. Also, physically, rabbits are larger animals with larger spleens that can produce more antibodies.

Rabbit monoclonal antibodies are similar to traditional mouse monoclonal antibodies while offering better specificity and sensitivity. Rabbits are immunized and the resulting spleen cells are fused with partner cells to make an immortal cell line that expresses antibodies. The antibodies are derived from a single clone and characterized for performance in applications. A clone or clones are then selected for antibody production.

As the rabbit natural repertoire is more diverse than the mouse, and the spleen is larger, their antibodies exhibit higher affinity for the antigen. Thus, rabbit monoclonal antibodies tend to give superior sensitivity in the application for which the clones were screened. An additional advantage of the rabbit diversity is that it allows for epitope recognition that may not be feasible with other systems. Other advantages include, natural diversity, high affinity and specificity, novel epitope recognition, cross-reactivity to human and mouse targets and ease of humanization. And, as provided herein, antibodies can be provided that show little to no interference with the therapeutic agents, such as other antibodies, peptides or small molecules.

A light or heavy chain variable region of an antibody has four framework regions interrupted by three hypervariable regions, known as complementary determining regions (CDRs). CDRs determine the specificity of antigen binding. The heavy chain and light chain each have three CDRs, designated from the N terminus as CDR1, CDR2, and CDR3 with the four framework regions flanking these CDRs. The amino acid sequences of the framework region are highly conserved and CDRs can be transplanted into other antibodies. Therefore, a recombinant antibody can be produced by combining CDRs from one or more antibodies with the framework of one or more other antibodies. Antibodies of the invention include antibodies that comprise at least one, two, three, four, five, or six (or combinations thereof) of the CDRs of any of the monoclonal antibodies described herein.

Polypeptides/antibodies of the invention comprise full-length rabbit anti-HER2/neu heavy chain variable regions, full-length rabbit light chain variable regions, binding fragments or variants thereof, and combinations thereof.
1B7 sequences:
Heavy chain (SEQ ID NO:1; CDR1, 2, and 3=SEQ ID NO:5, 6 and 7):
METGLRWLLLVAVLKGVQCQSVEESGGRLVTPGTPLTLTCTVSGIDLSDYAMGWVRQ APGKGLEYIGIISSSGNTHYARWARGRFTISKTSSTTVDLKMTSLTTEDTATYFCARNY PGYANYALWGQGTLVTVSSGQPKAPSVFPLAPCCGDTPSSTVTLGCLVKGYLPEPVT VTWNSGTLTNGVRTFPSVRQSSGLYSLSSVVSVTSSSQPVTCNVAHPATNTKVDKTVA PSTCSKPMCPPPELPGGPSVFIFPPKPKDTLMISRTPEVTCVVVDVSQDDPEVQFTWYI NNEQVRTARPPLREQQFNSTIRVVSTLPIAHQDWLRGKEFKCKVHNKALPAPIEKTIS KARGQPLEPKVYTMGPPREELSSRSVSLTCMINGFYPSDISVEWEKNGKAEDNYKTT PTVLDSDGSYFLYSKLSVPTSEWQRGDVFTCSVMHEALHNHYTQKSISRSPGK
Light chain (SEQ ID NO:2; CDR1, 2, and 3=SEQ ID NO:8, 9 and 10):
MDTRAPTQLLGLLLLWLPGATFARIVMTQTPASVSAAVGGTVTIKCQASESISNWLS WYQQKPGQPPKLLIYRASTLASGVPSRFSGSGSGTEYTLTISDLECADAATYYCQQDY IYNDIDNAFGGGTEVVVKGDPVAPTVLIFPPAADQVATGTVTIVCVANKYFPDVTVTW EVDGTTQTTGIENSKTPQNSADCTYNLSSTLTLTSTQYNSHKEYTCKVTQGTTSVVQS
FNRGDC
1G5 sequences:
Heavy chain (SEQ ID NO:3; CDR1, 2, and 3=SEQ ID NO:11, 12 and 13):
METGLRWLLLVAVLKGVQCQSLEESGGDLVKPGASLTLTCTASGFSFSSSAYMCWVR QAPGKGLEWIACIAAGSVGRTAYASWAKGRFTLSKISSTTVTLQMTSLTAADTATYFC ARGYAAYGGYDWPITYFKLWGPGTLVTVSSGQPKAPSVFPLAPCCGDTPSSTVTLGC LVKGYLPEPVTVTWNSGTLTNGVRTFPSVRQSSGLYSLSSVVSVTSSSQPVTCNVAHP ATNTKVDKTVAPSTCSKPMCPPPELPGGPSVFIFPPKPKDTLMISRTPEVTCVVVDVSQ DDPEVQFTWYINNEQVRTARPPLREQQFNSTIRVVSTLPIAHQDWLRGKEFKCKVHN KALPAPIEKTISKARGQPLEPKVYTMGPPREELSSRSVSLTCMINGFYPSDISVEWEKN GKAEDNYKTTPTVLDSDGSYFLYSKLSVPTSEWQRGDVFTCSVMHEALHNHYTQKS ISRSPGK
Light chain (SEQ ID NO:4; CDR1, 2, and 3=SEQ ID NO:14, 15 and 16):
MDTRAPTQLLGLLLLWLPGARCADIVMTQTPASVEAAVGGTVTIKCQASQSIYSGLA WYQQKPGQPPKLLIYDASDLPSGVPSRFKGSGSGTEFTLTISDLECADAATYYCQSYY GSSTTYGNTFGGGTEVVVKGDPVAPTVLIFPPAADQVATGTVTIVCVANKYFPDVTVT WEVDGTTQTTGIENSKTPQNSADCTYNLSSTLTLTSTQYNSHKEYTCKVTQGTTSVV QSFNRGDC

Heavy Chain CDRs

| | CDR- | | | CDR- | | | CDR- | | |
|---|---|---|---|---|---|---|---|---|---|
| Clone | H1 | Residues | Length | H2 | Residues | Length | H3 | Residues | Length |
| 1B7 | 49-53 | DYAMG | 5 | 68-83 | IISSSGNTHYARWARG | 16 | 115- | NYPGYANYAL | 10 |
| | | (SEQ ID | | | (SEQ ID NO: 6) | | 124 | (SEQ ID NO: 7) | |
| | | NO: 5) | | | | | | | |
| 1G5 | 49-54 | SSAYMC | 6 | 69-86 | CIAAGSVGRTAYASWAKG | 18 | 118- | GYAAYGGYDWPITYFKL | 17 |
| | | (SEQ ID | | | (SEQ ID NO: 12) | | 134 | (SEQ ID NO: 13) | |
| | | NO: 11) | | | | | | | |

Light Chain CDRs

| | CDR- | | | CDR- | | | CDR- | | |
|---|---|---|---|---|---|---|---|---|---|
| Clone | L1 | Residues | Length | L2 | Residues | Length | L3 | Residues | Length |
| 1B7 | 47-57 | QASESISNWLS | 11 | 73-79 | RASTLAS | 7 | 112- | QQDYIYNDIDNA | 12 |
| | | (SEQ ID NO: 8) | | | (SEQ ID NO: 9) | | 123 | (SEQ ID NO: 10) | |
| 1G5 | 47-57 | QASQSIYSGLA | 11 | 73-79 | DASDLPS | 7 | 112- | QSYYGSSTTYGNT | 13 |
| | | (SEQ ID NO: 14) | | | (SEQ ID NO: 15) | | 124 | (SEQ ID NO: 16) | |

CDR identification method per E. Kabat, T. Wu, H. Perry, Sequences of Proteins of Immunological Interest, fifth ed., US Department of Health and Human Services, National Institutes of Health, Bethesda MD, 1992.

A polypeptide variant, antibody variant or variant CDR differs by about, for example, 1, 2, 3, 4, 5, 6, 7, 8, 9, 10, 20, 30, 40, 50, 60 or more amino acid residues (e.g., amino acid additions, substitutions or deletions) from a polypeptide shown in SEQ ID NOs: 1-16 or a fragment thereof. Where this comparison requires alignment, the sequences are aligned for maximum homology. The site of variation can occur anywhere in the polypeptide. In one embodiment of the invention a variant polypeptide has activity substantially similar to a polypeptide shown in SEQ ID NOs: 1-16. Activity substantially similar means that when the polypeptide is used to construct an antibody, the antibody has the same or substantially the same activity/binding as the wild-type antibody.

As used herein, percent identity of two amino acid sequences (or of two nucleic acid sequences) is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al. (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the invention.

Identity or identical means amino acid sequence (or nucleic acid sequence) similarity and has an art recognized meaning. Sequences with identity share identical or similar amino acids (or nucleic acids). Sequence identity is the percentage of amino acids identical to those in the antibody's original amino acid sequence, determined after the sequences are aligned and gaps are appropriately introduced to maximize the sequence identity as necessary. Thus, a candidate sequence sharing 85% amino acid sequence identity with a reference sequence requires that, following alignment of the candidate sequence with the reference sequence, 85% of the amino acids in the candidate sequence are identical to the corresponding amino acids in the reference sequence, and/or constitute conservative amino acid changes.

The invention also includes polypeptide variants or CDR variants of SEQ ID NOs: 1-16. Polypeptide variants or CDR variants of SEQ ID NOs: 1-16 can comprise one or more amino acid substitutions, additions or deletions. In one embodiment, a variant polypeptide or variant CDR includes an amino acid sequence at least about 75% identical to a sequence shown as SEQ ID NOs: 1-16. In one embodiment, the variant polypeptide or CDR is at least about 75%, 80%, 85%, 90%, 95%, 98%, 99%, 99.5% or more identical to SEQ ID NOs: 1-16. Variant polypeptides or variant CDRs encode a variant antibody, which is an antibody comprising an amino acid sequence of SEQ ID NOs: 1-16 in which one or more amino acid residues have been added, substituted or deleted. For example, the variable region of an antibody can be modified to improve its biological properties, such as antigen binding. Such modifications can be achieved by e.g., site-directed mutagenesis, PCR-based mutagenesis, cassette mutagenesis. Variant antibodies comprise an amino acid sequence which is at least about 75%, 80%, 85%, 90%, 95%, 98%, 99%, 99.5% or more identical to the amino acid sequence of a heavy or light chain variable region of SEQ ID NOs: 1-16.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989)). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art.

The variant polypeptides can have conservative amino acid substitutions at one or more predicted non-essential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A polypeptide or antibody of the invention can be covalently or non-covalently linked to an amino acid sequence to which the polypeptide or antibody is not normally associated with in nature. Additionally, a polypeptide or antibody of the invention can be covalently or non-covalently linked to compounds or molecules other than amino acids. For example, a polypeptide or antibody can be linked to an indicator reagent (indicator reagents can include chromogenic agents, catalysts, such as enzyme conjugates, fluorescent compounds, such as fluorescein and rhodamine, chemiluminescent compounds, such as dioxetanes, acridiniums, phenanthridiniums, ruthenium, and luminol, radioactive elements, direct visual labels, as well as cofactors, inhibitors, magnetic particles, and the like; examples of enzyme conjugates include alkaline phosphatase, horseradish peroxidase, beta-galactosidase, and the like), an amino acid spacer, an amino acid linker, a signal sequence, a stop transfer sequence, a transmembrane domain, a protein purification ligand (e.g., glutathione-S-transferase, histidine tag, and staphylococcal protein A), or a combination thereof. In one embodiment of the invention a protein purification ligand can be one or more C amino acid residues at, for example, the amino terminus or carboxy terminus of a polypeptide of the invention. An amino acid spacer is a sequence of amino acids that are not usually associated with a polypeptide or antibody of the invention in nature. An amino acid spacer can comprise about 1, 5, 10, 20, 100, or 1,000 amino acids.

A polypeptide of the invention can be isolated from cells or tissue sources using standard protein purification techniques. Polypeptides of the invention can also be synthesized chemically or produced by recombinant DNA techniques. For example, a polypeptide of the invention can be synthesized using conventional peptide synthesizers.

A polypeptide of the invention can be produced recombinantly. A polynucleotide encoding a polypeptide of the invention can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system.

### Binding

Antibodies/binding portions thereof (antigen binding fragments) of the invention specifically bind HER2 (e.g. human HER2). “Specifically binds” means that the antibody recognizes and binds to HER2 with greater affinity than to other, non-specific molecules that are not HER2. For example, an antibody raised against an antigen (polypeptide) to which it binds more efficiently than to a non-specific antigen (e.g., a protein that is not related to or homologous to HER2) can be described as specifically binding to the antigen. Binding specificity can be tested using, for example, an enzyme-linked immunosorbant assay (ELISA), a radioimmunoassay (RIA), or a western blot assay using methodology well known in the art.

### Methods of Making Antibodies

Antibodies of the invention can be produced using methods known to those of skill in the art. For example, an HER2 antigen or a fragment thereof can be used to immunize animals, including rabbit. HER2 or a fragment thereof can be conjugated to a carrier protein and/or administered to the animals with an adjuvant. An HER2 antigen can comprise one or more epitopes (i.e., antigenic determinants). An epitope can be a linear epitope, sequential epitope or a conformational epitope. Epitopes within a polypeptide of the invention can be identified by several methods. See, e.g., U.S. Pat. No. 4,554,101; Jameson & Wolf, CABIOS 4:181-186 (1988). For example, HER2 can be isolated and screened. A series of short peptides, which together span the entire HER2 polypeptide sequence, can be prepared by proteolytic cleavage. By starting with, for example, 100-mer polypeptide fragments, each fragment can be tested for the presence of epitopes recognized in an ELISA. For example, in an ELISA assay an HER2 antigen, such as a 100-mer polypeptide fragment, is attached to a solid support, such as the wells of a plastic multi-well plate. A population of antibodies are labeled, added to the solid support and allowed to bind to the unlabeled antigen, under conditions where non-specific absorption is blocked, and any unbound antibody and other proteins are washed away. Antibody binding is detected by, for example, a reaction that converts a colorless substrate into a colored reaction product. Progressively smaller and overlapping fragments can then be tested from an identified 100-mer to map the epitope of interest.

Methods for preparing monoclonal antibodies from hybridomas are well known to those of skill in the art and include, e.g., standard cell culture methods and ascites production methods. Recombinant antibodies or fragments thereof produced by gene engineering can be made using the polynucleotide sequences of the invention. Genes encoding antibodies or fragments thereof can be isolated from hybridomas of the invention or other hybridomas. The genes can be inserted into an appropriate vector and introduced into a host cell. See, e.g., Borrebaeck & Larrick, Therapeutic Monoclonal Antibodies, Macmillan Publ. Ltd, 1990.

In one aspect, highly specific monoclonal antibodies were developed by immunizing rabbits, selecting spleenocytes and constructing commercial quantities of monoclonal antibodies suitable for clinical use. Most recombinant rabbit monoclonal antibodies are used only in research, so the use of monoclonal rabbit antibodies in the clinical space is unique. In addition, the antibodies described herein are superior for a number of reasons. For example, recombinant rabbit mAbs exhibit higher binding affinity to their ligand relative to recombinant mouse mAbs and, thereby, provide more reproducible results. Further, the rabbit monoclonal antibodies provided herein show limited/moderate to no therapeutic drug interference in the immunoassay.

Rabbit monoclonal antibodies (mAb) have been recognized for their advantages as research and diagnostic reagents: they have affinities 10-100 times higher than mouse mAbs; superior specificity that can distinguish even single amino acid differences and reduce cross-reactivity; broad epitope recognition that increases mAb diversity; great stability for consistent performance; and longer shelf life due to extra disulfide bonds in rabbit IgG (Feng L. et al. Am J Transl Res. 2011; 3(3):269-74; Rossi S. et al. American Journal of Clinical Pathology. 2005; 124(2):295-302; Vilches-Moure J G et al. J Vet Diagn Invest. 2005; 17(4):346-50).

To effectively discover specific mAbs, Yurogen uses its single B cell based SMab™ platform for efficient high-throughput screening for specific rabbit mAbs of interest. Briefly, one first enriches and sorts protien-recognizing B cells from splenocytes using fluorescence activated cell sorting (FACS); sorted cells are cultured and stimulated in 1 cell/well; positive clones are identified from single B cells using enzyme-linked immunosorbent assays (ELISA) and other desired assays against the protein of interest; supernatants and RNA are collected for future analysis; genes of naturally paired IgG light and heavy chains are then cloned from positive clones; and then one expresses and validates selected mAb clones. SMab™ platform routinely generates 300-500 testable clones of mAbs in 3-4.5 months, about 30% to 50% faster than traditional hybridoma and display platforms. Use of a large pool of splenocytes and scalable high-throughput design increases the diversity of the initial mAb pool recognizing the protein of interest. SMab™ platform delivers the earliest functional characterization of protein-specific mAbs using the culture supernatants from intermediate steps to reduce antibody development time by removing unnecessary workload.

### Conjugates

Antibodies of the invention can be covalently attached to other molecules such that covalent attachment does not affect the ability of the antibody to bind to HER2. For example, antibodies can be modified by, e.g., glycosylation, acetylation, pegylation, phosphorylation, amidation, derivatization by known protecting/blocking groups (e.g., methyl group, ethyl group, carbohydrate group), proteolytic cleavage, linkage to a cellular ligand or other protein.

Conjugated antibodies can be bound to various molecules including, for example, polymers, hyaluronic acid, fluorescent substances, luminescent substances, haptens, enzymes, metal chelates, cytotoxic agents, radionuclides, and drugs.

### Methods of Detection

One embodiment of the invention provides methods of detecting HER2 polypeptides in a sample. The methods comprise contacting the sample suspected of containing HER2 polypeptides with an antibody or antigen binding portion thereof of the invention to form HER2/antibody complexes. The presence of the HER2/antibody complexes are detected, thereby detecting the presence of the HER2 polypeptides.

The test sample can be, e.g., lymph node or tissue aspirate, serum, whole blood (for example, a blood test could consist of a postcard or cartridge that can test a drop of blood from a finger prick), plasma, circulating tumor cells, tumor cells or tissue (e.g., tissue biopsy) or ascites fluid. Polypeptide/antibody complexes can be detected by any method known in the art, including, but not limited to, enzyme-linked immunosorbent assay (ELISA), multiplex fluorescent immunoassay (MFI or MFIA), radioimmunoassay (RIA), sandwich assay, western blotting, immunoblotting analysis, an immunohistochemistry method, immunofluorescence assay, fluorescence-activated cell sorting (FACS) or a combination thereof.

An immunoassay for HER2 can utilize one antibody or several different antibodies. Immunoassay protocols can be based upon, for example, competition, direct reaction, or sandwich type assays using, for example, labeled antibody. Antibodies of the invention can be labeled with any type of label known in the art, including, for example, fluorescent, chemiluminescent, radioactive, enzyme, colloidal metal, radioisotope and bioluminescent labels

Antibodies of the invention or antigen-binding portions thereof can be bound to a support and used to detect the presence of HER2. Supports include, for example, glass, polystyrene, polypropylene, polyethylene, dextran, nylon, amylases, natural and modified celluloses, polyacrylamides, agaroses and magletite.

Antibodies of the invention can be used in a method of the diagnosis of a hyperproliferative disorder by obtaining a test sample from, e.g., a human or animal suspected of having a hyperproliferative disorder. The test sample is contacted with antibodies or antigen-binding portions thereof of the invention under conditions enabling the formation of antibody-antigen complexes (i.e., immunocomplexes). One of skill in the art is aware of conditions that enable and are appropriate for formation of antigen/antibody complexes. The amount of antibody-antigen complexes (including, for example, a complex of an antibody or antigen-binding portion thereof and HER2) can be determined by methodology known in the art. A level that is higher than that formed in a control sample indicates the presence of a hyperproliferative disorder. The amount of antibody/antigen complexes can be determined by methods known in the art.

A HER2 positive hyperproliferative disorder can be a neoplastic disorder including breast cancer, ovarian, stomach, adenocarcinoma of the lung, uterine cancer (such as uterine serous endometrial carcinoma), gastric cancer and/or salivary duct carcinoma.

The antibodies/assays described herein can be used to identify and monitor patients having tumors that overexpress HER2 and, thus, are candidates for targeted drug treatment. The antibodies/assays described herein show limited to no interference with therapeutic agents. Accordingly, the immunoassays described herein fill an unmet need in the breast cancer care space.

The immunoassays described herein can be used to test for HER2 positive breast cancer, monitor the serum levels of HER2 in patients receiving drug therapy, to detect recurrence, or detect HER2 disease in women that tested tissue HER2 negative. The immunoassays described herein can be used to detect an elevated or rising level of serum HER2 in a woman, which can indicate the appearance of HER2 disease in women that were thought to be HER2-negative (e.g., by tissue testing). The immunoassays described herein also can be used in conjunction with measuring circulating tumor cells (CTC) or as an adjunct to identify, or help identify, patients that are in need of, or would benefit from, a positron emission tomography (PET) scan.

The following are provided for exemplification purposes only and are not intended to limit the scope of the invention described in broad terms above.

### Lateral Flow Assays (LFAs)

A lateral flow assay (LFA) is based on the movement of a liquid sample though a polymeric strip with attached molecules that interact with the analyte, providing a signal that can be visually detected. LFA is generally a paper-based platform for the detection and/or quantification of analytes (such as proteins, haptens, nucleic acids and amplicons) in what are often complex mixtures, where the sample is placed on a test device and the results are displayed within about 5-30 min, such 5-10 minutes. Low development costs and ease of production of LFAs have resulted in the expansion of its applications to multiple fields in which rapid tests are needed, such as biomedicine, agriculture, food and environmental sciences. LFA-based tests are widely used in hospitals, physician's offices and clinical laboratories for the qualitative and quantitative detection of specific antigens and antibodies, as well as products of gene amplification, in such settings as veterinary medicine, quality control, product safety in food production, and environmental health and safety, including to screen for animal and human diseases, pathogens, chemicals, toxins and water pollutants, among others.

For LFA, a liquid sample (such as urine, saliva, sweat, serum, plasma, whole blood and other fluids) containing the analyte of interest moves without the assistance of external forces (capillary action) through various zones of polymeric strips, on which molecules that can interact with the analyte are attached. A typical lateral flow test strip can consist of overlapping membranes that are mounted on a backing card for better stability. The sample is applied at one end of the strip, on the adsorbent sample pad, which can be loaded with buffer salts and surfactants that make the sample suitable for interaction with the detection system. The sample migrates through the conjugate release pad, which contains antibodies that are specific to the target analyte and are conjugated to colored or fluorescent particles-such as colloidal gold and latex microspheres (depending on the elements of recognition used, LFAs can be categorized into different types, such as ‘lateral flow immunoassays’ (LFIAs), in which antibodies are used as recognition elements, and nucleic acid LFA (NALFA), in which the detection of amplicons which can be formed during the polymerase chain reaction (PCR) are used). The sample, together with the conjugated antibody bound to the target analyte, migrates along the strip into the detection zone. This is generally a porous membrane (usually composed of nitrocellulose) with specific biological components (mostly antibodies or antigens) immobilized in lines. Their role is to react with the analyte bound to the conjugated antibody. Recognition of the sample analyte results in an appropriate response on the test line, while a response on the control line indicates the proper liquid flow through the strip. The read-out, represented by the lines appearing with different intensities, can be assessed by eye or using a dedicated reader (device).

Provided herein is a point-of-care multiple diagnostic assay with multiple test lines allowing the rapid and simultaneous detection of multiple analytes present in samples, including, for example, HER2 positive hyperproliferative disorders such as a neoplastic disorders including breast cancer, ovarian, stomach, adenocarcinoma of the lung, uterine cancer (such as uterine serous endometrial carcinoma), gastric cancer and/or salivary duct carcinoma, providing a powerful toll for cancer detection and progression, for example, before, after and/or during treatment. In order to test multiple analytes simultaneously under the same conditions, additional test lines of antibodies specific to different analytes can be immobilized in an array format. On the other hand, multiple test lines loaded with the same antibody can be used for semi-quantitative assays. The principle of this ‘ladder bars’ assay is based on the stepwise capture of colorimetric conjugate-antigen complexes by the immobilized antibody on each successive line, where the number of lines appearing on the strip is directly proportional to the concentration of the analyte. The liquid flows across the device because of the capillary force of the strip material and, to maintain this movement, an absorbent pad can be attached at the end of the strip. The role of the absorbent pad is to wick the excess reagents and prevent backflow of the liquid. A current example of an LFA is a pregnancy test stick.

Two formats of the LFIA can be distinguished: direct and competitive. A direct test is used for larger analytes such as the p24 antigen used in the human immunodeficiency virus (HIV) test as well as analytes with multiple antigenic sites such as human chorionic gonadotropin (hCG) used in pregnancy tests. The hCG test is an example of a sandwich-based assay, where the target is immobilized between two complementary antibodies. In the direct test, the presence of the test line indicates a positive result and the control line usually contains species-specific anti-immunoglobulin antibodies, specific for the antibody in the particular conjugate. In the case of small molecules with single antigenic determinants, which cannot bind to two antibodies simultaneously, competitive tests are used. In this type of test, the analyte blocks the binding sites on the antibodies on the test line, preventing their interactions with the colored conjugate. Therefore, a positive result is indicated by the lack of signal in the test line, while the control line should be visible independently of the test result.

As for a label colloidal gold is a widely used label in commercial LFIA. Another popular label is latex, which can be tagged with a variety of detector reagents such as colored or fluorescent dyes, and magnetic or paramagnetic components. As latex can be produced in multiple colors, it has an application in multiplex assays, which require discrimination between numerous lines. Carbon and fluorescent labels, or enzymatic modification of the labels, are also used. Carbon nanotubes, fluorescent labels, quantum dots, upconverting phosphors can all be used as labels. Another detection system that can be used is FACTT, an acronym for a sensitive protein detection system whereby amplification of the detection mAb occurs when coupled with T7 polymerase. Rather than measuring the mAb directly, the reader detects RNA molecules generated by the polymerase, thus greatly amplifying the result. This test can result in a qualitative color change but may also benefit from a reader (device). It may take 20-30 minutes.

There are many advantageous to using such an assay including, for example, point-of-care, providing inexpensive, rapid and easy tests desirable in many industries/countries and because of their long shelf life and the fact that refrigeration is often not required for storage, these tests are well suited for use in developing countries, small ambulatory care settings, remote regions and battlefields. Further as the visual result is usually clear, no additional equipment is needed; however, an optional device can be used for the readout.

### Examples

### Example I: Validation Summary for Serum HER2 ELISA Laboratory-Developed Test

### A. Reportable Range

### Materials and Methods

Experiments were performed to determine the span of test result values over which the accuracy of the assay's measurements can be established. Only results that fall within the reportable, or linear, range were quantitatively reported. The boundaries of the reportable range were defined by the upper and lower limits of quantification (ULOQ and LLOQ). The final calculated concentration for an analyte in a sample should be the same using any dilution that falls within the range of the assay.

### Study Design

The Reportable Range specification was addressed by developing a robust standard curve using two replicates per concentration, with seven concentrations across the anticipated analytical measuring range (AMR), and closely fitting a curve using polynomial regression analysis.

The highest and lowest standard points were established as the ULOQ and LLOQ by determining the coefficient of variation (CV) of the reportable unit (concentration in ng/ml) in twenty separate assays, with CV defined as the standard deviation (SD) divided by the mean concentration, and expressed as a percentage.

A linearity assay was utilized to further evaluate the reportable range. Three natural samples with suitable HER2 levels were diluted 1:25, 1:100 and 1:200, in addition to the 1:50 dilution specified in the assay's standard operating procedure (SOP). After measurement, linearity was demonstrated by quantifying these values as a percentage of the expected HER2 concentration for each tested dilution.

SoftMax Pro 7 Data Analysis Software (Molecular Devices) was used to help determine all values used in the validation summary. Values are reported using three decimal places.

### Acceptance Criteria

For the standard curve:
The mean value for the plate blank (Lm1-Lm2, where Lm1 equals OD measurement at 450 nm and Lm2 equals OD plate correction at 630 nm) must be less than 0.150 OD, indicating low non-specific background.
The mean value for the highest standard concentration must be greater than 1.500 OD following plate blank subtraction, signifying a robust assay.
The standard curve fit must use a regression coefficient R² between 0.990 and 1.000, demonstrating strong value prediction.

For the limits of quantification:
The determined ULOQ and LLOQ must have CV values of less than 20% for acceptance.

For the linearity assay:
Ranges between 80% and 120% of the expected value indicate acceptable linearity.

### Results

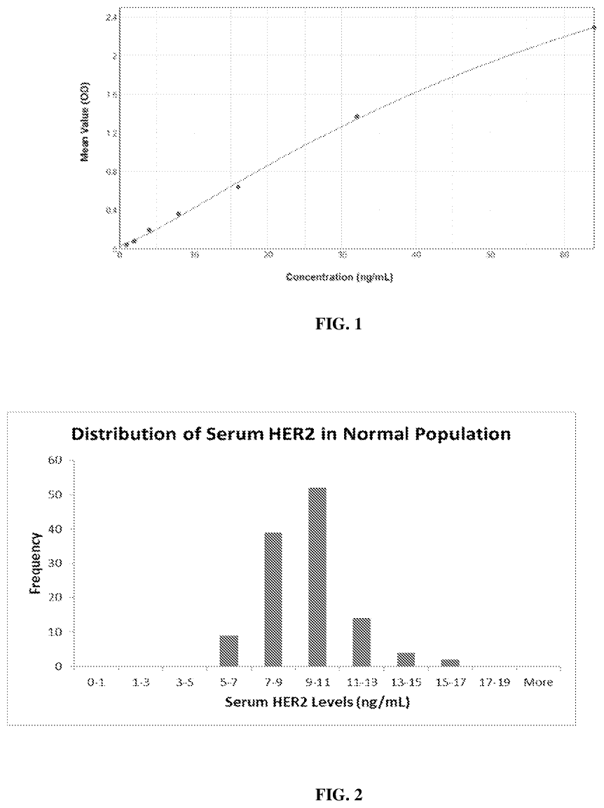

FIG. 1 shows a representative standard curve for the serum HER2 ELISA based on the data shown in Table 1. As indicated in Table 1, the seven standard curve points were 64, 32, 16, 8, 4, 2 and 1 ng/mL. The plate blank was 0.030 OD; the corrected high standard was 2.287 OD; and the regression coefficient R² of the 4-parameter logistic standard curve fit was 0.999.

TABLE 1
Standard Curve

| ng/mL | OD | OD mean | OD final |
|---|---|---|---|
| 0 | 0.028 | 0.030 | 0 |
| | 0.032 | | |
| 1 | 0.067 | 0.068 | 0.038 |
| | 0.069 | | |
| 2 | 0.109 | 0.108 | 0.078 |
| | 0.107 | | |
| 4 | 0.222 | 0.223 | 0.193 |
| | 0.223 | | |
| 8 | 0.392 | 0.391 | 0.361 |
| | 0.389 | | |
| 16 | 0.664 | 0.668 | 0.638 |
| | 0.672 | | |
| 32 | 1.400 | 1.396 | 1.366 |
| | 1.393 | | |
| 64 | 2.320 | 2.317 | 2.287 |
| | 2.314 | | |

For the upper limit of quantification (ULOQ), the measured concentrations for the 64 ng/ml high standard point had a CV of 0.4% in twenty tests. This concentration was, therefore, acceptable for use. Similarly, the twenty 1 ng/ml low standard point measurements had a CV of 15.9% and, consequently, this concentration was acceptable for use as the lower limit of quantification (LLOQ). See Table 2.

TABLE 2
Upper and Lower Limits of Quantification

| Sample | Low Standard (ng/mL) | High Standard (ng/mL) |
|---|---|---|
| 1 | 0.974 | 64.048 |
| 2 | 1.088 | 64.031 |
| 3 | 1.090 | 63.981 |
| 4 | 1.060 | 63.982 |
| 5 | 0.962 | 63.971 |
| 6 | 1.153 | 64.260 |
| 7 | 1.164 | 64.230 |
| 8 | 0.937 | 64.661 |
| 9 | 0.848 | 63.975 |
| 10 | 0.902 | 64.015 |
| 11 | 1.079 | 64.045 |
| 12 | 1.139 | 64.866 |
| 13 | 0.552 | 64.170 |
| 14 | 0.905 | 63.967 |
| 15 | 0.988 | 64.100 |
| 16 | 1.160 | 64.024 |
| 17 | 0.725 | 63.917 |
| 18 | 1.041 | 63.963 |
| 19 | 0.912 | 63.903 |
| 20 | 1.143 | 63.964 |
| Tests | 20 | 20 |
| Mean (ng/mL) | 0.991 | 64.104 |
| Max (ng/mL) | 1.164 | 64.866 |
| Min (ng/mL) | 0.552 | 63.903 |
| SD (ng/mL) | 0.158 | 0.247 |
| CV | 15.9% | 0.4% |

Linearity was well within acceptable tolerances for all three natural samples tested. See Table 3. For the 1:25, 1:100 and 1:200 dilutions, serum A95 gave corrected values of 103.6%, 100.3% and 109.6% of the expected concentrations, respectively; serum B2 gave corrected values of 114.2%, 103.1% and 105.2%; and serum B4 gave corrected values of 96.9%, 92.8% and 95.3%.

TABLE 3
Linearity

| | 1:50 dilution | 1:25 dilution | 1:100 dilution | 1:200 dilution |
|---|---|---|---|---|
| Sample | (ng/mL) | (ng/mL) | (ng/mL) | (ng/mL) |
| A95 | 6.171 | 12.788 | 3.094 | 1.691 |
| B2 | 7.462 | 17.046 | 3.848 | 1.962 |
| B4 | 9.014 | 17.469 | 4.184 | 2.148 |

Percentage of Expected Concentration

| | | | | |
|---|---|---|---|---|
| A95 | 100.0% | 103.6% | 100.3% | 109.6% |
| B2 | 100.0% | 114.2% | 103.1% | 105.2% |
| B4 | 100.0% | 96.9% | 92.8% | 95.3% |

### DISCUSSION

Overall, the serum HER2 ELISA described herein met acceptability requirements for standard curve, upper and lower limits of quantification, and linearity. The AMR for this assay was 1.0-64.0 ng/mL.

### B. Analytical Sensitivity

Experiments were performed to define the ability of the assay to detect very low concentrations of its target in a biological sample. Often referred to as the limit of detection (LOD), it is the lowest actual concentration of analyte in a specimen that can be consistently detected, but not necessarily quantified. Most often, the LOD resides below the linear range of an assay but cannot be higher than the LLOQ. The LOD can be determined statistically, by calculating the point at which a signal can be distinguished from background.

### Materials and Methods

A statistical determination of analytical sensitivity was obtained. Using one microtiter plate, 80 measurements were utilized alongside a complete standard curve: 40 measurements of the blank, and 40 measurements of the lowest standard (1.0 ng/mL). A common formula for the LOD was used (see, for example, Burd, 2010, Clin Microbiol Rev, 23:550-76), followed by calculation of the corresponding concentration as follows:

LOD=(the mean of the blank) plus (1.65× the standard deviation of the blank) plus (1.65× the standard deviation of the lowest non-zero standard). The determined LOD calculation must be below the established LLOQ of the ELISA.

### Results

Mean of blank (OD): 0.031
Standard deviation of blank (OD): 0.003
Standard deviation of lowest standard (OD): 0.003
LOD (OD)=0.031+(1.65×0.003)+(1.65×0.003)
LOD (OD)=0.031+0.005+0.005
LOD (OD)=0.041
LOD (ng/mL)=0.049

### DISCUSSION

The LOD calculation of analytical sensitivity for the serum HER2 ELISA was 0.049 ng/ml, well below the LLOQ of 1.0 ng/mL. The analytical sensitivity of the ELISA described herein, therefore, was acceptable.

### C. Precision

Experiments were performed to determine how well a given measurement could be reproduced when the test was applied repeatedly to multiple aliquots of a single homogeneous sample. It was further defined as the closeness of agreement between independent results obtained under stipulated conditions, and as random analytical error, caused by factors that vary during normal operation of the assay. Precision was expressed based on statistical measurements of imprecision, including standard deviation (SD) and coefficient of variation (CV).

The types of relevant precision measures are repeatability (within-run precision; same operator, same time, etc.) and reproducibility (run-to-run precision; different operators, different days, etc.).

### Materials and Methods

Repeatability. Three samples of high, medium and low HER2 concentrations, based upon the reportable range of the assay, were used. Each sample was tested twenty times on one plate in order to determine the mean, SD and CV percentage for each sample (where CV equals SD divided by mean). One operator was used.

Reproducibility. The same three samples above were tested in twenty separate assays, in duplicate. The precision was expressed in CV percentage for each sample, using the mean and SD determined for each sample concentration. Two operators were used.

Three HER2 controls also were formulated and defined at high, medium and low concentrations to further gauge reproducibility during assay measurements. Each control concentration was tested ten times using two operators, to define their acceptable ranges.

The precision acceptance requirements were determined in CV percentage (CV %), and were less than 10% for repeatability, and less than 20% for reproducibility. 5

### Results

For repeatability, results with the three samples were as follows: high concentration CV=2.0%; medium concentration CV=1.2%; and low concentration CV=3.9%. All determined CVs were well below the acceptable maximum CV of 10.0%. See Table 4.

TABLE 4
Repeatability

| | High | Medium | Low |
|---|---|---|---|
| | Concentration | Concentration | Concentration |
| Sample | (ng/mL) | (ng/mL) | (ng/mL) |
| 1 | 33.648 | 9.432 | 2.034 |
| 2 | 34.344 | 9.161 | 1.938 |
| 3 | 33.461 | 9.093 | 1.915 |
| 4 | 34.105 | 9.125 | 1.844 |
| 5 | 33.039 | 9.276 | 1.932 |
| 6 | 32.777 | 9.218 | 1.868 |
| 7 | 32.733 | 9.175 | 1.909 |
| 8 | 33.359 | 9.091 | 1.800 |
| 9 | 33.582 | 9.100 | 1.918 |
| 10 | 33.197 | 9.139 | 1.883 |
| 11 | 32.782 | 8.940 | 1.812 |
| 12 | 33.599 | 9.028 | 1.921 |
| 13 | 32.556 | 9.204 | 1.958 |
| 14 | 32.913 | 9.055 | 1.932 |
| 15 | 32.798 | 9.125 | 1.897 |
| 16 | 33.370 | 9.118 | 1.806 |
| 17 | 31.694 | 9.161 | 1.987 |
| 18 | 32.023 | 8.978 | 1.897 |
| 19 | 33.881 | 9.247 | 2.065 |
| 20 | 33.304 | 9.276 | 2.037 |
| Tests | 20 | 20 | 20 |
| Mean (ng/mL) | 33.158 | 9.147 | 1.918 |
| Max (ng/mL) | 34.344 | 9.432 | 2.065 |
| Min (ng/mL) | 31.694 | 8.940 | 1.800 |
| SD (ng/mL) | 0.653 | 0.112 | 0.074 |
| CV | 2.0% | 1.2% | 3.9% |

For reproducibility, results with the three samples were as follows: high concentration CV=11.2%; medium concentration CV=12.9%; and low concentration CV=17.8%. All determined CVs were below the acceptable maximum CV of 20.0%. See Table 5.

TABLE 5
Reproducibility

| | High | Medium | Low |
|---|---|---|---|
| | Concentration | Concentration | Concentration |
| Sample | (ng/mL) | (ng/mL) | (ng/mL) |
| 1 | 32.915 | 9.755 | 1.948 |
| 2 | 33.158 | 9.147 | 1.918 |
| 3 | 37.615 | 9.586 | 2.006 |
| 4 | 31.667 | 6.835 | 1.782 |
| 5 | 37.500 | 7.621 | 1.979 |
| 6 | 32.881 | 8.959 | 2.282 |
| 7 | 25.899 | 8.410 | 3.127 |
| 8 | 33.810 | 7.942 | 1.829 |
| 9 | 30.337 | 8.960 | 2.303 |
| 10 | 32.926 | 11.303 | 1.797 |
| 11 | 35.765 | 8.586 | 1.927 |
| 12 | 32.387 | 7.692 | 1.794 |
| 13 | 35.188 | 9.970 | 2.026 |
| 14 | 32.768 | 7.156 | 1.581 |
| 15 | 35.670 | 8.240 | 1.736 |
| 16 | 28.886 | 6.857 | 1.434 |
| 17 | 32.192 | 8.478 | 1.956 |
| 18 | 38.460 | 7.917 | 1.694 |
| 19 | 25.231 | 8.659 | 1.778 |
| 20 | 27.800 | 8.778 | 2.104 |
| Tests | 20 | 20 | 20 |
| Mean (ng/mL) | 32.653 | 8.543 | 1.950 |
| Max (ng/mL) | 38.460 | 11.303 | 3.127 |
| Min (ng/mL) | 25.231 | 6.835 | 1.434 |
| SD (ng/mL) | 3.656 | 1.103 | 0.347 |
| CV | 11.2% | 12.9% | 17.8% |

For controls, a range in expected values was determined by taking the mean of ten measurements and adding or subtracting approximately two standard deviations, which represented a 95% confidence interval. For Control A, the expected range was 26 to 40 ng/ml; for Control B, the expected range was 8.5 to 15.0 ng/ml; and for Control C, the expected range was 4.2 to 8.2 ng/mL. See Table 6.

TABLE 6
Controls

| | Control A | Control B | Control C |
|---|---|---|---|
| Sample | (ng/mL) | (ng/mL) | (ng/mL) |
| 1 | 34.687 | 12.586 | 6.448 |
| 2 | 32.98 | 10.672 | 4.881 |
| 3 | 28.033 | 9.334 | 4.99 |
| 4 | 31.677 | 11.081 | 5.968 |
| 5 | 33.705 | 13.265 | 5.995 |
| 6 | 34.926 | 13.671 | 7.254 |
| 7 | 28.568 | 9.37 | 4.861 |
| 8 | 33.213 | 12.963 | 6.805 |
| 9 | 27.45 | 12.307 | 7.479 |
| 10 | 39.044 | 13.936 | 7.148 |
| Tests | 10 | 10 | 10 |
| Mean (ng/mL) | 32.4283 | 11.9185 | 6.1829 |
| Max (ng/mL) | 39.044 | 13.936 | 7.479 |
| Min (ng/mL) | 27.45 | 9.334 | 4.861 |
| SD (ng/mL) | 3.609036623 | 1.702016924 | 1.008998233 |
| Range (mean ± 2SD) | 26-40 ng/mL | 8.5-15.0 ng/mL | 4.2-8.2 ng/mL |

### DISCUSSION

The serum HER2 ELISA meets acceptability requirements for both the repeatability and reproducibility measurements of precision. In addition, three controls were established: Control A (above the reference interval of the assay); Control B (near the reference interval cutoff); and Control C (below the reference interval). These controls were included with every ELISA to assist with monitoring precision during clinical testing.

### D. Analytical Specificity

Experiments were performed to define the ability of the assay described herein to detect only the intended target, and to determine that quantification of the target was not affected by cross-reactivity from related or potentially interfering specimen-related conditions. Interfering substances refer to the effect that a compound other than the analyte in question had on the accuracy of measurement. The two aspects of analytical specificity evaluated were cross-reactivity and interference.

Analytical specificity studies were performed following Clinical and Laboratory Standards Institute (CLSI) EP07-A2 (2005, “Interference Testing in Clinical Chemistry; Approved Guideline-Second Edition”).

### Cross-Reactivity

The following four recombinant human (rh) proteins (three family members related to HER2 and one unrelated protein) were tested individually at the elevated concentration of 200 ng/ml in the assay alongside the standard curve: rhEGFR, rhHER3, rhHER4, and rhPD-L1 (each provided by Sino Biological). Cross-reactivity was assessed by calculating the percentage of measured recombinant protein concentration versus the loaded initial concentration of 200 ng/mL.

### Interference

Three methods were utilized. In each, interference was assessed by calculating the recovery percentage from the initial concentration:

First, the related four proteins at the same elevated concentration of 200 ng/ml were individually added to a midpoint rhHER2 concentration (considered the reference point). Following the assay run, the measured HER2 concentration was compared to the expected concentration, and percent recovery was calculated.

Second, the following five therapeutic antibodies were run for interference in serum samples: trastuzumab (Herceptin; Roche), pertuzumab (Perjeta; Roche), pembrolizumab (Keytruda; Merck); nivolumab (Opdivo; Bristol-Myers Squibb); and atezolizumab (Tecentriq; Roche). Each drug was spiked at the physiologically relevant concentration of 100 μg/mL into an endogenous sample with a known assay measurement (a natural reference point). Herceptin and Perjeta were additionally tested in combination. Any changes from the expected concentration were determined, and percent recovery was calculated.

Third, the same five therapeutic antibodies (Herceptin, Perjeta, Keytruda, Opdivo and Tecentriq) were added to a midpoint rhHER2 concentration and run in the assay to evaluate their interference with the HER2 standard. Each drug was spiked at the physiologically relevant concentration of 100 μg/mL, any concentration changes determined, and the percent recovery was calculated.

For cross-reactivity, any recombinant protein found to generate values greater than 5.0% of that expected for HER2 was considered cross-reacting. For interference, any protein or therapeutic antibody that, when present, generated a measured concentration less than 80% or greater than 120% of that expected for HER2 was considered interfering.

### Results

All four recombinant proteins tested did not cross-react in the HER2 ELISA at 200 ng/ml, meeting acceptability requirements. See Table 7

TABLE 7
Recombinant Protein Cross-Reactivity

| rhEGFR (ng/mL) | rhHER3 (ng/mL) | rhHER4 (ng/mL) |
|---|---|---|
| 0.000 | 0.000 | 0.000 |

Percentage of Cross-Reactivity

| | | |
|---|---|---|
| 0.0% | 0.0% | 0.0% |

Recovery percentages were 102.9% for rhEGFR, 107.4% for rhHER3, 120.9% for rhHER4 and 95.2% for rhPD-L1. Therefore, the rhEGFR, rhHER3 and rhPD-L1 recombinant proteins showed no interference with the HER2 ELISA at 200 ng/ml, and results met acceptability requirements. The rhHER4 recombinant protein showed slight positive interference in the HER2 ELISA under these same conditions. See Table 8.

TABLE 8
Recombinant Protein Interference

| Standard | and rhEGFR | and rhHER3 | and hrHER4 |
|---|---|---|---|
| (ng/mL) | (ng/mL) | (ng/mL) | (ng/mL) |
| 5.220 | 5.370 | 5.608 | 6.310 |

Percentage of Expected Concentration

| | | | |
|---|---|---|---|
| 100.0% | 102.9% | 107.4% | 120.9% |

Average recovery percentages were 91.3% for Herceptin, 73.0% for Perjeta, 72.4% for Herceptin/Perjeta, 93.1% for Keytruda, 91.0% for Opdivo and 92.8% for Tecentriq (the experiment was performed using five seras to confirm results). Therefore, four therapeutic antibody drugs, Herceptin, Keytruda, Opdivo and Tecentriq, when tested alone, showed no interference with the HER2 ELISA at 100 μg/mL. Perjeta, either alone or in combination with Herceptin, moderately interfered with the ELISA under these same conditions.

An antibody-drug conjugate derived from Herceptin, trastuzumab emtansine (Kadcyla; Roche) was also tested for potential interference at 100 μg/mL in two serum samples (designated MM3 and F62C). The average recovery percentage in these two seras was 92.2% for Kadcyla, indicating an acceptable level of interference. See Table 9.

TABLE 9
Drug Interference with Serum

| | | and | and | and | and | and | and |
|---|---|---|---|---|---|---|---|
| | sHER2 | Herceptin | Perjeta | H/P | Keytruda | Opdivo | Tecentriq |
| Sample | (ng/mL) | (ng/mL) | (ng/mL) | (ng/mL) | (ng/mL) | (ng/mL) | (ng/mL) |
| MM2 | 13.427 | 12.064 | 9.699 | 9.856 | 12.904 | 12.458 | 12.581 |
| A91 | 8.176 | 7.562 | 5.902 | 5.944 | 7.593 | 7.517 | 7.571 |
| D1 | 8.586 | 7.718 | 6.290 | 6.328 | 7.782 | 7.714 | 8.150 |
| 26113-12 | 11/164 | 10.458 | 8.256 | 7.931 | 10.555 | 10.250 | 10.417 |
| 26113-13 | 8.738 | 7.897 | 6.405 | 6.239 | 7.971 | 7.747 | 7.821 |
| Percentage of | | | | | | | |
| Expected | | | | | | | |
| Concentration | | | | | | | |
| MM2 | 100% | 89.9% | 72.2% | 73.4% | 96.1% | 92.8% | 93.7% |
| A91 | 100% | 92.5% | 72.2% | 72.7% | 92.9% | 91.9% | 92.6% |
| D1 | 100% | 89.9% | 73.3% | 73.7% | 90.6% | 89.8% | 94.9% |
| 26113-12 | 100% | 93.7% | 74.0% | 71.0% | 94.5% | 91.8% | 93.3% |
| 26113-13 | 100% | 90.4% | 73.3% | 71.4% | 91.2% | 88.7% | 89.5% |
| Average | | 91.3% | 73.0% | 72.4% | 93.1% | 91.0% | 92.8% |

Recovery percentages were 90.8% for Herceptin, 70.1% for Perjeta, 99.6% for Keytruda, 121.1% for Opdivo, 97.8% for Tecentriq. Therefore, the antibody drugs Herceptin, Keytruda and Tecentriq showed no interference with the HER2 ELISA standard at 100 μg/mL, and results met acceptability requirements. The drug, Opdivo, showed slight positive interference, while Perjeta moderately interfered with the HER2 ELISA standard under these same conditions. See Table 10.

TABLE 10
Drug Interference with Standard

| | and | and | and | and | and |
|---|---|---|---|---|---|
| Standard | Herceptin | Perjeta | Keytruda | Opdivo | Tecentriq |
| (ng/mL) | (ng/mL) | (ng/mL) | (ng/mL) | (ng/mL) | (ng/mL) |
| 14.431 | 13.105 | 10.122 | 14.373 | 17.481 | 14.114 |

Percentage of Expected Concentration

| | | | | | |
|---|---|---|---|---|---|
| 100.0% | 90.8% | 70.1% | 99.6% | 121.1% | 97.8% |

### DISCUSSION

The serum HER2 ELISA exhibited no cross-reactivity with related protein family members rhEGFR, rhHER3 and rhHER4, or with rhPD-L1. Of these protein family members, only rhHER4 exhibited slight positive recombinant protein interference in the assay. The tested therapeutic drugs trastuzumab (Herceptin), trastuzumab emtansine (Kadcyla), pembrolizumab (Keytruda), nivolumab (Opdivo) and atezolizumab (Tecentriq) also failed to interfere in the HER2 ELISA when run with patient seras. Of these drugs, only Opdivo showed slight positive interference with the HER2 ELISA standard, while the others exhibited no standard interference in the assay. However, pertuzumab (Perjeta) moderately interfered with both serum samples and the HER2 ELISA standard, indicating that care should be taken when measuring HER2 concentrations in samples containing this therapeutic antibody.

### E. Accuracy (Trueness)

To determine the closeness of the agreement between the results of a single measurement and the true value of the analyte. Two primary methods were used to evaluate accuracy: a recovery study and a comparison-of-methods study.

Recovery studies test whether the assay can measure the analyte of interest when a known amount is present in the intended specimen matrix. Samples were constructed for testing by adding known amounts of analyte to patient specimens. The amount of analyte recovered was then compared to the amount added to the specimen.

Comparison-of-methods studies examine the assay's results with another currently existing method.

### Materials and Methods

### Study Design

For the recovery study, two different spiked concentrations of rhHER2 within the assay range, approximately 10 and 20 ng/mL, were added to two normal serum samples with known HER2 concentrations. The average recovery percentages were determined to gauge assay trueness.

For the comparison-of-methods study, the serum HER2 values of five patient seras were compared using ELISA results with two operators, and the HER2 quantitative ELISA using Bayer Centaur/ACS methodology (LabCorp).

### Acceptance Criteria

The difference between the average recovery and 100% recovery was used to judge test acceptability for the recovery study. Determined HER2 concentrations must be greater than 80% and less than 120% of the expected recoveries for both spiked rhHER2 concentrations.

For the comparison-of-methods study, the average CV % between this HER2 assay and the LabCorp assay must be less than 20% for each operator.

### Results

For Spike 1, recoveries were 105.6% and 91.9% in the two female samples, F27A and F85. For Spike 2, recoveries were 105.3% and 96.2% in the same two female samples. Table 11.

TABLE 11
Recovery

| | Spike 1 | Spike 2 | Spike 1 Recovery | Spike 2 Recovery |
|---|---|---|---|---|
| Sample | (ng/mL) | (ng/mL) | (%) | (%) |
| Spike | 9.752 | 18.924 | | |
| F27A | 10.296 | 19.929 | 105.6% | 105.3% |
| F85 | 8.964 | 18.203 | 91.9% | 96.2% |

For Operator 1, the average CV for the five serum samples, MM1-MM5, in comparison with their LabCorp results was 12.8%. For Operator 2, the average CV for these samples in comparison with their LabCorp results was 15.2%. Table 12.

TABLE 12
Comparison-of-Methods

| | LabCorp | Operator | Operator 1 | Operator 2 | Operator 2 |
|---|---|---|---|---|---|
| Sample | (ng/mL) | (ng/mL) | CV % | (ng/mL) | CV % |
| MM1 | 14.4 | 12.613 | 9.355 | 11.429 | 16.267 |
| MM2 | 12.4 | 10.330 | 12.879 | 11.308 | 6.514 |
| MM3 | 16.4 | 13.202 | 15.278 | 13.181 | 15.389 |
| MM4 | 7.9 | 5.730 | 22.515 | 5.96 | 19.794 |
| MM5 | 7.1 | 6.729 | 3.794 | 5.511 | 17.819 |
| Average | | | 12.8 CV % | | 15.2 CV % |

### DISCUSSION

With determined percentage recoveries of 91.9, 96.2, 105.3 and 105.6%, the recovery experiment demonstrated an acceptable accuracy range within 80 to 120% for the serum HER2 ELISA. Similarly, with average CV percentages of 12.8 and 15.2% for both operators, the comparison-of-methods experiments also demonstrated acceptable accuracy for this ELISA.

### F. Reference Interval

To determine the range of the test for a normal population (e.g., the range of values typically found in individuals who do not have the disease or condition that is being assayed by the test). For quantitative assays, the reference interval is often reported as a value below a specific measurement or concentration.

Materials and Methods 120 normal seras gathered from a variety of sources were run in the assay. Since roughly 99% of breast cancers occur in women, only female seras were included. Samples with any known interfering substances were excluded. The resulting data was then statistically evaluated to determine the normal range of the test. The normal range was quantitatively defined as the mean value of these 120 samples plus or minus two standard deviations.

Ranges were calculated using R statistics package (Version 3.5.2) using all 120 serum samples. Each sample must show less than 20% CV between duplicate measurements for inclusion.

### Acceptance Criteria

The reference interval for the serum HER2 ELISA was defined between the lower reference limit (the 5th percentile) and the upper reference limit (the 95th percentile) of the determined normal distribution.

### Results

FIG. 2 and Table 13 shows the reference range for the serum HER2 ELISA. The generated data was approximately normal in distribution, as the values for mean (9.601 ng/mL) and median (9.522 ng/mL) were extremely close, and the slightly positive distribution skew (0.839) was less than 1.0. Therefore, using the formula mean±2SD, the normal reference range for the above HER2 distribution equaled 5.853 to 13.349 ng/mL.

For clinical purposes, the serum HER2 reference range determination is one-sided, or less than 13.3 ng/ml, using one decimal place for reporting patient results. Table 13.

TABLE 13
Distribution and Summary Statistics for HER2 Reference Range.

| | Mean | Median | SD | Min | Max | |
|---|---|---|---|---|---|---|
| N | (ng/mL) | (ng/mL) | (ng/mL) | (ng/mL) | (ng/mL) | Skew |
| 120 | 9.601 | 9.522 | 1.874 | 5.770 | 16.337 | 0.839 |

### DISCUSSION

Applying a 95% confidence range to the determined distribution results of 120 normal female serum samples defined the reference interval for the serum HER2 ELISA as less than 13.3 ng/mL.

### Example II: Immunogen Exposure

The extracellular domain (ECD) of the human HER2 protein was expressed in HEK 293F cells. Two New Zealand White rabbits were immunized with the recombinantly-expressed HER2 ECD using multi-point subcutaneous injection. Complete Freund's adjuvant (CFA) and incomplete Freund's adjuvant (IFA) were used to boost each rabbit's immunization response against the HER2 ECD protein, with the final boost before spleen harvest being delivered via intravenous (iv) injection.

### Example III: B-Cell Isolation and Screening

B cells were isolated by grinding the spleen. B cells against HER2 ECD protein were enriched using magnetic activated cell sorting (MACS) and fluorescence activated cell sorting (FACS). Single B cells were deposited in individual wells of a 96-well culture plates and cultured for about 2 weeks. At the end of culture, supernatants from individual B cell clones were used to screen for HER2 ECD-positive clones using ELISA. B cells from positive clones were harvested for RNA preparation, cDNA synthesis, and PCR amplification of paired IgG heavy chain and light chain sequences.

### Example IV: Recombinant Engineering of Light and Heavy Chain Clones, Co-Expression, Production and Purification

VH and VL fragments of paired IgG genes from individual positive clones were assembled into mammalian expression plasmids. Expression plasmids containing paired heavy and light chains genes were sequenced and validated. Afterwards, plasmids of paired heavy and light chain genes were co-transfected into 293F cells for expressing recombinant rabbit IgG protein. IgG proteins in 293F culture supernatants were harvested for purification by protein-A affinity chromatography.

### Example V: Structural Information

All rabbit IgGs were gamma 1 heavy chain paired with kappa 1 light chain. Purity was confirmed using SDS-PAGE. The heavy and light chains from clone 1B7 were determined to have the sequences shown above (SEQ ID NOs: 1, 2 and 5-10), and the heavy and light chains from clone 1G5 were determined to have the sequences shown above (SEQ ID NOs: 3, 4 and 11-16). Table 14 shown below provides the molecular weight (MW) and pI for each clone.

TABLE 14

| Antibody | MW* | pI* |
|---|---|---|
| 1B7 | | |
| Heavy | 49,956.82 Da | 8.68 |
| Light | 25,317.34 Da | 4.67 |
| 1G5 | | |
| Heavy | 50,782.95 Da | 8.78 |
| Light | 25,213.20 Da | 4.67 |

*theoretical

### Example VI: Serum HER2-Neu ELISA Assay

The HERTEST™ soluble HER2-neu immunoassay is a solid-phase sandwich ELISA designed to measure human HER2 protein present in serum samples. The immunoassay utilizes a rabbit monoclonal antibody for capture and a different biotinylated rabbit monoclonal antibody for detection. Both capture and detection reagents specifically bind to different regions of the extracellular domain of the HER2 protein.

### Materials and Methods

To perform the test, the capture antibody was immobilized on the interior surface of the wells of a microtiter plate. Standards, controls and samples were pipetted into the wells, and any HER2 present was bound by the immobilized antibody. After washing away any unbound proteins, the immobilized HER2 is then detected with the biotinylated detection antibody. The amount of detection antibody bound to HER2 was measured with a streptavidin/horseradish peroxidase conjugate. Following a wash to remove unbound conjugate, a substrate solution was added to the wells, and color developed in proportion to the amount of HER2 bound. After color development was stopped, the absorbance was measured using a microplate spectrophotometer.

### Controls and Calibration

A standard curve was performed each day of patient testing. One standard curve was suitable for any number of microtiter plates run simultaneously, but a blank microtiter plate was included on every run.

Controls were generated by spiking normal human serum samples at pre-determined concentrations of sHER2 protein. Based on internal validations, the following control ranges were used: Control A: 26.0 to 40.0 ng/ml; Control B: 8.5 to 15.0 ng/ml; and Control C: 4.2 to 8.2 ng/mL. Patient (all female) reference range was 0-13.3 ng/mL.

### Specimen Type and Stability

Refrigerated sera was tested within 14 days of collection. Frozen serum was stable for up to 1 year when maintained at 80° ° C. To reduce the potential for protein breakdown, multiple freeze-thaw cycles were avoided by generating multiple aliquots upon the receipt of refrigerated serum or at the time of the first thaw.

### Specimen Handling

For samples received refrigerated, refrigeration was maintained until testing, unless testing was to be delayed longer than 7 days. If testing was to be delayed longer than 7 days, or the time since specimen collection was to exceed 14 days, labeled Eppendorf snap-cap tubes were prepared for aliquots of 200 μL of each sample and stored in the −80° C. freezer until testing was performed.

For samples received frozen, samples were maintained frozen. For testing, samples were thawed at room temperature, gently vortexed to mix, pipetted from the parent tube for testing and then labeled Eppendorf snap-cap tubes were prepared for aliquots of 200 μL and stored frozen at −80° C.

### Interfering Substances

Recombinant human proteins were prepared at 200 ng/mL and assayed using HERTEST™. The following exhibited no cross-reactivity or interference: EGFR, HER3, HER4 or PD-L1.

Therapeutic drugs were added to serum samples at 100 mg/mL. The following exhibited no interference using HERTEST™: Opdivo (nivolumab), Keytruda (pembrolizumab), Tecentriq (atezolizumab) or Herceptin (trastuzumab). The following exhibited interference using HERTEST™: Perjeta (pertuzumab).

### Sample Rejection

Serum specimens with gross hemolysis or gross lipemia, or serum samples received at room temperature or warmer, were not tested with HERTEST™. Multiple freeze-thaw cycles of samples were avoided.

### Protocol

Plate Coating

The number of wells (or strips) necessary to perform the patient testing was determined, and 1 mL of 1×PBS (Invitrogen 10×PBS, ThermoFisher #AM9625) was generated for each well or strip. Enough Capture Antibody (1B7; Yurogen) to coat each well at 1 μg/mL was prepared in an appropriately sized conical tube using the formula: C1V1=C2V2 (Concentration 1×Volume 1=Concentration 2×Volume 2). For example, if C1=1.5 for antibody 1B7, VI is unknown, C2 is 1, and V2 is the number of strips (e.g., 5), then (1.5)(x)=(1)(5), and x=3.33 μL of 1B7 capture antibody into 5 mL of 1×PBS. The conical tubes were vortexed (VWR Analog Vortex) to thoroughly mix the capture solution, and the contents were poured into a clean reagent reservoir. 100 μL of Capture Antibody were pipetted into each well (or strip). Each well (or strip) was covered with an adhesive plate cover and incubated at 2-8° C. overnight (˜16 hours).

Standard curve aliquots 1-7 were made by combining 968 μL of Sample Buffer and 32 μL of the rhHER2 (FLAG) (Recombinant Human ErbB2/Her2-neu (FLAG), Sino Biological Inc., #10004-HCCH) stock (2 μg/mL) into a labeled 1.5 mL Eppendorf snap-cap tube, making 1000 μL of STD1 (64 ng/mL). Using STD1, 1:2 serial dilutions were used to create all 7 standard concentrations (e.g., 500 μL of STDI was combined with 500 μL of sample buffer to make STD2 (32 ng/ml); 500 μL of STD2 was combined with 500 μL of sample buffer to make STD3 (16 ng/mL); these steps were repeated to make STD4 (8 ng/ml), STD5 (4 ng/ml), STD6 (2 ng/mL), STD7 (1 ng/mL)). Standard curve samples were stored at −80° C.

### ELISA

1.5 L of Wash Buffer (0.05% PBST [0.05% Tween 20 in 1×PBS]) was prepared for each plate. 0.5 mL of Tween20 (Sigma, #P1379) was added to each liter of 1×PBS and mixed well. A coated plate (Costar ELISA plates (VWR, #29442-302)) was washed on a plate washer (BioTek ELx50 or Molecular Devices AquaMax 2000). After washing, any remaining fluid was removed from the plate (e.g., by decanting, by blotting with paper towels, etc.), and 200 μL of Blocking Buffer (Casein in PBS, ThermoFisher #37528) was pipetted into each well. The wells were covered with an adhesive plate and incubated at room temperature for 1 hour.

Sample Buffer/Antibody Diluent (10% Casein in 1×PBS) was prepared (˜40 mL/plate). After incubation, plates were washed on a plate washer as described herein. 245 μL of Sample Buffer then was pipetted into a labeled snap-cap tube for each patient sample to be tested, as well as for each standard and control. A 1:50 dilution of each patient sample as well as each standard and control was generated (e.g., 5 μL of each sample in 95 μL of Sample Buffer). 100 μL of each sample (patient, standard, control, and blank (i.e., 100 μL of Sample Buffer)) was pipetted into their respective wells as indicated on the plate map. The wells were covered with an adhesive plate cover, placed in a microplate shaker (Corning LSE Digital Microplate Shaker) at 500 rpms for 1 min, and then incubated at room temperature for 1 hour.

Detection Antibody (1G5-Biotinylated; Yurogen) was prepared just before use (e.g., within 30 mins) using Antibody Diluent at 0.25 μg/mL using the same formula described above. After incubation, plates were washed on a plate washer as described herein. Detection Antibody was vortexed briefly and poured into a clean reagent reservoir. 100 μL was pipetted into each well and the plate was covered with an adhesive plate cover. Each plate was placed in the microplate shaker at 500 rpms for 1 min and then incubated at room temperature for 1 hour. Conjugate (SA-HRP) (Streptavidin HRP (BD Biosciences #554066)) was prepared in Antibody Diluent at a 1/5000 dilution in a clean conical tube just before use (e.g., within 15 minutes) according to Table 15.

TABLE 15

| μL HRP | mL Sample Diluent | Plate Fill |
|---|---|---|
| 2 | 10 | Full |
| 1 | 5 | Half |
| 0.5 | 2.5 | ¼ |

After incubation, plates were washed on a plate washer as described herein. The conjugate was vortexed briefly, and the contents poured into a clean reagent reservoir. 100 μL of the conjugate was pipetted into each well, and the wells were covered with an adhesive plate cover. The plate was placed in the microplate shaker at 500 rpms for 1 min and then incubated at room temperature for 30 mins. After incubation, plates were washed on a plate washer as described herein. The TMB Substrate (ThermoFisher #34021) was prepared immediately before use in a clean conical tube according to Table 16.

TABLE 16

| mL TMB | mL Peroxide | Plate Fill |
|---|---|---|
| 5 | 5 | Full |
| 2.5 | 2.5 | Half |
| 1.25 | 1.25 | ¼ |

The substrate was vortexed briefly and the contents poured into a clean reagent reservoir. 100 μL of the substrate was pipetted into each well, and the wells were covered with an adhesive plate cover. The plate was placed in the microplate shaker at 500 rpms for 1 min, and then incubated at room temperature for 20 minutes in the dark.

### Data Analysis

After incubation, 100 μL of Stop Solution (H₂SO₄ (FisherChemical #SA215-1)) was pipetted into each well. The uncovered plate was placed into a VersaMax plate reader (Molecular Devices VersaMax Microplate reader) and SoftMax Pro software (SoftMax Pro Microplate reader software (7.0.3)) was started. SoftMax Pro 7 Data Acquisition and Analysis Software calculated and reported the following quality control parameters (if the below criteria were not met, the run was deemed invalid and was repeated): the mean OD value for the plate blank (Lm1-Lm2) should be <0.150; the mean OD value for the highest standard concentration should be >1.500 following blank subtraction; the correlation coefficient R² calculation for the standard curve should fall between 0.995 and 1.000; the results with Controls A, B and C should fall within their expected concentration ranges (Control A: 26 to 40 ng/ml; Control B: 8.5 to 15 ng/ml; and Control C: 4.2 to 8.2 ng/ml).

The following are representative settings that were used for the SoftMax Pro software: Lm1=450 nm; Lm2=630; m (plate reference); Shake 5 s; Standards concentrations in “ng/mL”; Background (reference) was subtracted: (Lm1!-Lm2!); Standard Curve: 4-Parameter Logistic, linear coordinates.

### Example VII: Drug Interference Data with Antibody Pairs

Capture antibodies were coated onto high-binding plates overnight at 4° C. The next day, plates were washed, blocked with casein for 1 hr at room temperature and washed again. Serum or rhHER2 protein was combined with 100 μg/ml of each drug (i.e., Herceptin, Perjeta, Keytruda, Opdivo, Tecentriq) for 30 min prior to plating (1 hr at room temp; positive interference controls were created by incubating matched non-biotinylated detection antibodies (100 μg/ml) with analyte). After incubation, plates were washed, followed by the addition of biotinylated detection antibody and incubated for 1 hr at room temp. Following a wash, SA-HRP was added for 30 min at room temp. After a final wash, TMB was added for 20 min at room temp in the dark. The reaction was stopped with H₂SO₄ and protein concentration was determined using a VersaMax reader and SoftMax Pro software. See Tables 17, 18 and 19 for the results.

TABLE 17

| Serum | Herceptin | Perjeta | Keytruda | Control |
|---|---|---|---|---|
| MM2(1B7/1G5biotin) | 89.30% | 74.10% | 92.10% | 18.60% |
| A72(1B7/1G5biotin) | 89.20% | 76.60% | 96.40% | 18.20% |
| MM2(1B7/2A3biotin) | 75.10% | 62.90% | 76.60% | 13.70% |
| A72(1B7/2A3biotin) | 83.50% | 70.00% | 88.00% | 10.60% |
| MM2(2A3/1G5biotin) | 92.00% | 64.10% | 95.00% | 16.10% |
| A72(2A3/1G5biotin) | 85.10% | 63.10% | 94.20% | 15.60% |

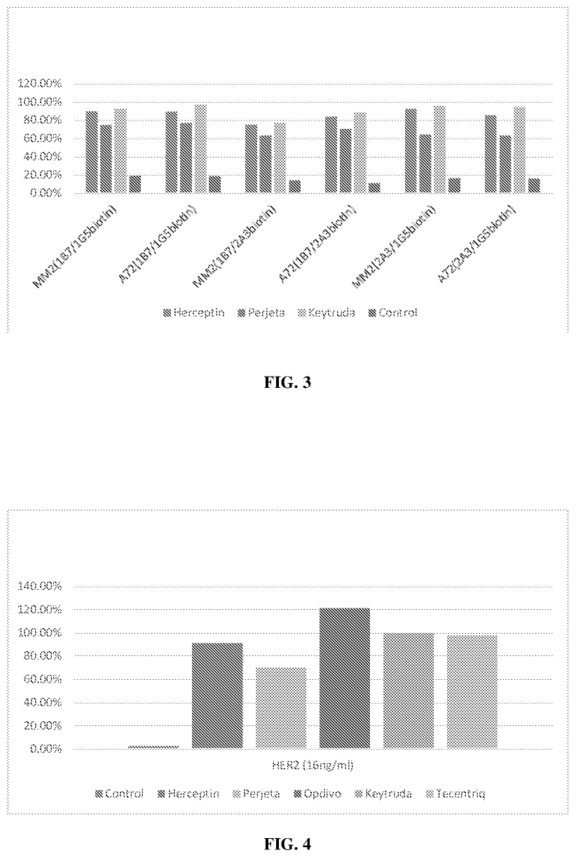

The results in Table 17 are graphically shown in FIG. 3. FIG. 3 is a graph showing drug interference with 1B7/1G5-biotin antibody pair or IB7/2A3-biotin antibody pair. As can be seen in FIG. 3 (and Table 17), 1B7/IG5-biotin pair showed no (or minimal) interference with Herceptin or Keytruda and modest interference with Perjeta.

TABLE 18

| | Control | Herceptin | Perjeta | Opdivo | Keytruda | Tecentriq |
|---|---|---|---|---|---|---|
| HER2 (16 ng/ml) | 2.55% | 90.80% | 70.10% | 121.10% | 99.60% | 97.80% |

The results shown in Table 18 are graphically represented in FIG. 4. FIG. 4 (and Table 18) demonstrates no interference with Herceptin, Keytruda or Tecentriq, positive interference with Opdvio and moderate negative interference with Perjeta.

TABLE 19

| Patient | | | | | | | Herceptin / |
|---|---|---|---|---|---|---|---|
| Designation | Control | Herceptin | Perjeta | Keytruda | Opdivo | Tecentriq | Perjeta |
| 26113-MO1-B1 | 9.33% | 87.80% | 73.30% | 91.80% | 92.40% | 94.60% | 75.20% |
| 26113-12-B1 | 2.26% | 93.70% | 74.00% | 94.50% | 91.80% | 93.30% | 71.00% |
| 26113-13-B1 | 0.00% | 90.40% | 73.30% | 91.20% | 88.70% | 89.50% | 71.40% |
| A91 | 4.44% | 92.50% | 72.20% | 92.90% | 91.90% | 92.60% | 72.70% |
| RC1 | 2.77% | 89.90% | 73.30% | 90.60% | 89.80% | 94.90% | 88.50% |

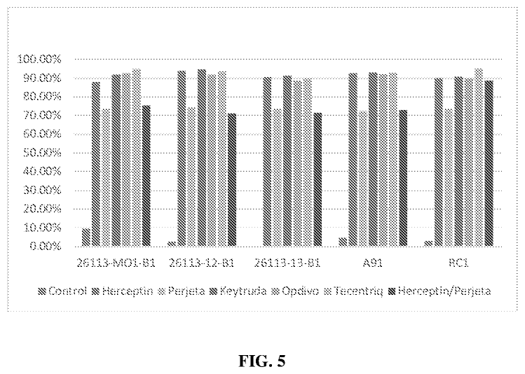

The results shown in Table 19 are graphically shown in FIG. 5. FIG. 5 (and Table 19) demonstrates no interference with Herceptin, Keytruda, Opdvio, or Tecentriq, and moderate negative interference with Perjeta and Herceptin/Perjeta combination.

All publications, nucleotide and amino acid sequence identified by their accession nos., patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. A method to detect HER2 polypeptides or fragments thereof in a sample comprising:
(a) analyzing a biological sample using a lateral flow immunoassay (LFA), wherein the LFA comprises at least one anti-HER2 rabbit monoclonal antibody or binding fragment thereof selected from the group consisting of:
(i) an anti-HER2 rabbit monoclonal antibody comprising a heavy chain variable domain (VH) comprising the amino acid sequence of SEQ ID NO:1 and a light chain variable domain (VL) comprising the amino acid sequence of SEQ ID NO:2;
(ii) an anti-HER2 rabbit monoclonal antibody comprising a heavy chain variable domain (VH) comprising the amino acid sequence of SEQ ID NO:3 and a light chain variable domain (VL) comprising the amino acid sequence of SEQ ID NO:4;
(iii) an anti-HER2 rabbit monoclonal antibody or binding fragment thereof comprising a heavy chain and a light chain, wherein the heavy chain comprises three CDR regions having the amino acid sequence SEQ ID NO: 5, 6 and 7; and the light chain comprises three CDR regions having the amino acid sequence SEQ ID NO: 8, 9 and 10 and
(iv) an anti-HER2 rabbit monoclonal antibody or binding fragment thereof comprising a heavy chain and a light chain, wherein the heavy chain comprises three CDR regions having the amino acid sequence SEQ ID NO: 11, 12 and 13; and the light chain comprises three CDR regions having the amino acid sequence SEQ ID NO: 14, 15 and 16
and
(b) detecting polypeptide/antibody complexes, wherein the detection of polypeptide/antibody complexes is an indication that the HER2 polypeptide is present in the sample.

2. The method of claim 1, wherein the LFA comprises at least 2 anti-HER2 antibodies.

3. The method of claim 1, wherein the LFA comprises (i) and (ii).

4. The method of claim 1, wherein the LFA comprises (iii) and (iv).

5. The method of claim 1, wherein the sample is lymph node or tissue aspirate (e.g., breast), serum, whole blood, plasma, urine, saliva, tears, cerebrospinal fluid, supernatant from normal cell lysates, supernatant from pre-neoplastic cell lysates, supernatant from neoplastic cell lysates and/or supernatants from carcinoma cell lines maintained in tissue culture.

6. The method of claim 1, wherein the lateral flow assay comprises a detectable label, wherein the label is detectable by visual inspection.

7. The method of claim 6, wherein the label detectable by visual inspection comprises gold colloidal particles.

8. The method of claim 1, wherein the sample is obtained from a subject that is being treated with a therapeutic agent.

9. The method of claim 8, wherein the therapeutic agent is at least one of trastuzumab, trastuzumab emtansine, pembrolizumab, pertuzumab, nivolumab, atezolizumab or a combination thereof.

10. The method of claim 8, further comprising (c) determining how a patient is responding to treatment based on the results in (b).

11. The method of 1, wherein two or more different analytes are detected.

12. The method of claim 11, wherein the analytes comprise HER2 and at least one analyte selected from the group consisting of a small molecule drug or therapeutic agent, a cancer antigen, an antibody (e.g., an antibody in response to a bacterial or viral infection or a treatment antibody), nucleic acids and/or proteins.
